(19) United States
(12) Patent Application Publication
(10) Pub. No.: US 2010/0096096 A1
(43) Pub. Date: Apr. 22, 2010

(54) USE OF AN ADDITIVE FOR THE PRODUCTION OF DECORATIVE PAPER

(76) Inventors: Jose Luis Egiburu, Tolosa (ES); Juan Antonio Navalpotro, Tolosa (ES)

Correspondence Address: Thomas M. Galgano, 20 W. Park Avenue, Suite 204, Long Beach, NY, 11561, US

(21) Appl. No.: 12/531,337

(22) PCT Filed: Apr. 30, 2007
(86) PCT No.: PCT/EP07/03802

§ 371 Date: Sep. 15, 2009

(51) Int. Cl. D21H 17/52; C07C 43/04; C08G 65/04

(52) U.S. Cl. 162/164.3; 568/618; 162/158; 528/421; 528/419

(57) ABSTRACT

The additive includes a polymer of an alkylene glycol or a copolymer of different alkylene glycols.

The invention relates to a process for the production of a decorative paper sheet which is more readily impregnable with the thermosetting resins used in the decorative laminate industry.

Decorative laminates have been manufactured throughout the world for a substantial number of years and have been utilized not only for the manufacture of furniture but also doors, desk tops, countertops, wall panels, flooring and various other items.

There are two main types of decorative laminates: “high pressure laminates” and “low pressure laminates”.

High pressure laminates are composite products which consist of a stack of up to 20 paper layers. The outer layer is melamine-formaldehyde resin (or mixtures of said resin with other amino resins) impregnated decorative paper, and the core consists of phenol-formaldehyde resin impregnated unbleached kraft papers. This assembly is consolidated under heat (around 140° C.) and pressure (70 to 95 bars) for 30 to 50 min.

In low pressure laminates, only one decorative paper saturated in melamine-formaldehyde resin (or mixtures of said resin with other amino resins) is used. The impregnated sheet is directly pressed on board surfaces. Typically, pressing time varies from 15 to 25 seconds, pressure between 20 to 27 bars, and temperature between 170 and 190° C.

Decorative papers used to manufacture such laminates can be used as produced in the papermaking machine, that is, as plain colors, or, additionally, they can be printed with any design (woods, stones, fantasies, . . . ) before impregnation.

The impregnable decorative paper is generally produced with cellulose fibers, predominantly hardwoods (80% to 100% of total fibers) combined with minor amounts of softwoods (0% to 20% of total fibers). Pigments (such as titanium dioxide, iron oxides, organic pigments, . . . ) and fillers are added in amounts up to 100% based on total fibers to obtain the required color and opacity. Aluminum salts for pH adjustment, as well as wet-strength resins to make it possible the paper impregnation in aqueous thermosetting resins, are also used.

Decorative papers must be highly absorbent with respect to the thermosetting resin with which they will be saturated.

For uniform and fast flow of the thermosetting resin into the sheet, the paper must be hydrophilic and porous. Paper porosity is mainly determined by the refining of cellulose fibers. This treatment has also a big impact on other paper properties such as strength, capillary absorption, resin absorption capacity and dimensional stability.

Certain level of refining is needed to get good formation, paper strength as well as efficient retention of pigments and fillers.

In general, impregnation properties can be improved by reducing refining as a more porous structure can be generated. However, this has a deleterious effect on paper strength, formation, as well as retention of fibers, pigments and fillers during the papermaking process.

It is an object of the present invention to provide a method of producing high porosity decorative paper while keeping enough refining for good paper formation, strength and optimal retention of fibers, pigments and fillers.

Another object of the invention is to disclose a method for producing decorative paper with reduced wet expansion.

Another object of the invention is to provide a method of producing printing base decorative paper with diminished surface closure after printing which makes the printed decorative paper more readily impregnable.

All these objects of the invention are achieved by the use of an additive including a polymer of an alkylene glycol or a copolymer of different alkylene glycols for the production of decorative paper.

According to a preferred embodiment, the additive includes polypropylene glycol or a copolymer of ethylene glycol and propylene glycol.

According to a preferred embodiment, the additive includes polypropylene glycol with a molecular weight between 100 g/mol and 10,000 g/mol, preferably between 2,000 g/mol and 4,000 g/mol. According to a preferred embodiment, the additive includes poly(ethylene glycol-co-propylene glycol) with a molecular weight between 100 g/mol and 10,000 g/mol, preferably between 2,000 g/mol and 5,000 g/mol. The ethylene glycol/propylene glycol ratio in the copolymer is from 99/1 to 1/99, preferably between 50/50 and 1/99.

According to a preferred embodiment, the additive is added to the cellulosic fibrous furnish before sheet forming on the paper machine wire.

The additive may be added continuously or discontinuously.

According to a preferred embodiment, the additive is incorporated in bulk to the cellulosic fibrous furnish in quantities between 0.05% and 3.0% by weight, preferably between 0.25% and 2% by weight, referred to the total solids content.

According to another preferred embodiment, the additive is applied to the surface of the decorative paper. The additive may be applied by coating or by impregnation.

According to another preferred embodiment, the decorative paper is impregnated (soaked) with the additive.

The use of the before-mentioned additive is a modification of the process for the production of decorative paper. Subject of the invention, however, is also the decorative paper produced thereby.

According to the invention, certain polyalkylene glycol type polymers or copolymers are included in the decorative paper composition.

More specifically, polypropylene glycol of different molecular weights can be used for this purpose. Block or random copolymers of propylene glycol and ethylene glycol or other alkylene glycols can be used, too. Graft copolymers of these polyalkylene glycol type polymers or copolymers onto other acrylic or vinylic polymers could also be utilized.

In a particular case, the decorative paper sheet comprises from 0.1% to 3% (referred to total solids content) of polypropylene glycol, preferably from 0.25% to 2%. Molecular weight of such a polymer is from 100 to 10,000 g/mol, preferably from 2,000 to 4,000 g/mol. The polymer is added to a furnish containing cellulose, titanium dioxide, colored pigments, fillers, wet strength resin and aluminum salts. This furnish is used to prepare handsheets according to the Rapid Köthen method. The basis weight of the handsheet having such composition is from 50 to 150 g/m², preferably from 70 to 100 g/m². The ash content can vary from 20% to 45%, preferably from 25% to 40%. In comparison to the standard paper (that made without polypropylene glycol), the decorative handsheets obtained according to this recipe have higher porosity (Gurley value decrease up to 40%), increased absorbency (Klemm capillary absorption increase up to 20%), and decreased resin penetration time (resin penetration time decrease up to 20%).

In another particular case, the decorative paper sheet comprises from 0.1% to 3% (referred to total solids content) of poly(ethylene glycol-co-propylene glycol), preferably from 0.25% to 2%. The copolymer molecular weight is between 100 g/mol and 10,000 g/mol, preferably between 2,000 g/mol and 5,000 g/mol. The ethylene glycol/propylene glycol ratio in the copolymer is from 99/1 to 1/99, preferably between 50/50 and 1/99.

The copolymer is added to a furnish containing cellulose, titanium dioxide, colored pigments, fillers, wet strength resin and aluminum salts. This furnish is used to prepare handsheets according to the Rapid Köthen method. The basis weight of the handsheet having such composition is from 50 to 150 g/m², preferably from 70 to 100 g/m². The ash content can vary from 20% to 45%, preferably from 25% to 40%. In comparison to the standard paper (that made without poly(ethylene glycol-co-propylene glycol)), the decorative handsheets obtained according to this recipe have higher porosity (Gurley value decrease up to 20%), increased absorbency (Klemm capillary absorption increase up to 10%), and decreased resin penetration time (resin penetration time decrease up to 17%).

In one advantageous embodiment, the decorative paper is produced in a Fourdrinier paper machine. The decorative paper sheet comprises from 0.05% to 1.0% (referred to total solids content) of polypropylene glycol, preferably from 0.1% to 0.3%. Molecular weight of such polymer is around 2,000 g/mol. The polymer is added to a furnish containing cellulose, titanium dioxide, colored pigments, fillers, wet strength resin and aluminum salts. The polymer can be added in the mixing chest in discontinuous way, or at the constant part in continuous dosification. The decorative paper having such composition has a basis weight from 40 to 150 g/m², preferably from 70 to 100 g/m². The ash content can vary from 20% to 45%, preferably from 25% to 40%. In comparison to the standard paper (that made without polypropylene glycol), the decorative paper sheet obtained according to this recipe has higher porosity (Gurley value decrease up to 60%), increased absorbency (Klemm capillary absorption increase up to 35%), decreased resin penetration time (resin penetration time decrease up to 50%), decreased wet expansion (cross direction wet expansion decrease up to 18%).

Another embodiment is related to a decorative printing base paper produced in a Fourdrinier paper machine. The decorative paper sheet comprises from 0.05% to 1.0% (referred to total solids content) of polypropylene glycol, preferably from 0.1% to 0.3%. Molecular weight of such polymer is around 2,000 g/mol. The polymer is added to a furnish containing cellulose, titanium dioxide, colored pigments, fillers, wet strength resin and aluminum salts. The polymer can be added in the mixing chest in discontinuous way, or at the constant part in continuous dosification. The decorative printing base paper having such composition has a basis weight from 50 to 150 g/m², preferably from 60 to 100 g/m². The ash content can vary from 25% to 45%, preferably from 30% to 40%. In comparison to the standard paper (that made without polypropylene glycol), the decorative printing base paper sheet obtained according to this recipe has higher porosity (Gurley value decrease up to 21%), increased absorbency (Klemm capillary absorption increase up to 10%), decreased wet expansion (cross direction wet expansion decrease up to 13%), slightly decreased smoothness

(Bekk smoothness decrease up to 6%) but similar printability when compared to the standard grade. In addition, paper closure after printing is lower with the paper made of the modified composition described in the invention.

The polyalkylene glycol type polymers and their copolymers of this invention can also be included in the decorative paper via surface treatment with water dispersions of such polymers or via impregnation of decorative paper in said dispersions.

In a particular case, decorative paper made in a Fourdrinier papermaking machine from a suspension of cellulose, titanium dioxide, colored pigments, fillers, wet strength resin and aluminum salts, is impregnated in polypropylene glycol dispersions. Molecular weight of polypropylene glycol is from 100 to 10,000 g/mol, preferably from 2,000 to 4,000 g/mol. Decorative paper is impregnated in polypropylene glycol aqueous dispersions and dried. Said polymer concentration is adjusted so that the final polymer content in the decorative paper is between 0.1% and 0.5%. Decorative paper sheet treated as described has lower wet expansion (cross direction wet expansion decrease up to 5%).

### EXAMPLE 1

This example shows the effect of polypropylene glycol or poly(ethylene glycol-co-propylene glycol) in laboratory handsheets (based on DIN EN ISO 5269-2) for four different furnish compositions.

Furnish A: 75 g of eucalyptus pulp were disintegrated in 2 liters of water. The pulp suspension was then diluted with 2 liters of water. 10 ml of NaOH 3% solution and 60 g of titanium dioxide were added to the pulp suspension. The mixture was thoroughly stirred with a high speed pigment disintegrator for 10 minutes. Then, under gentle stirring, 2.7 g of polyaluminum chloride and 3.7 g of wet strength resin were added. With this furnish, laboratory handsheets in around 75 g/m² were prepared using the Rapid Köthen method and were used as a reference of the standard recipe.

To evaluate the effect of the addition of polypropylene glycol (molecular weight around 2,000 g/mol), other furnishes were prepared in the same way, adding variable quantities of said polymer after the wet strength resin.

The results are shown in Table 1.

Furnish B: 75 g of eucalyptus pulp were disintegrated in 2 liters of water. The pulp suspension was then diluted with 2 liters of water. 10 ml of NaOH 3% solution and 67.5 g of titanium dioxide were added to the pulp suspension. The mixture was thoroughly stirred with a high speed pigment disintegrator for 10 minutes. Then, under gentle stirring, 2.7 g of polyaluminum chloride and 3.7 g of wet strength resin were added. With this furnish, laboratory handsheets in around 80 g/m² were prepared using the Rapid Köthen method and were used as a reference of the standard recipe.

To evaluate the effect of the addition of polypropylene glycol (molecular weight around 2,000 g/mol), other furnishes were prepared in the same way, adding variable quantities of said polymer after the wet strength resin.

The results are shown in Table 2.

Furnish C: 75 g of eucalyptus pulp were disintegrated in 2 liters of water. The pulp suspension was then diluted with 2 liters of water. 10 ml of NaOH 3% solution and 52.5 g of titanium dioxide were added to the pulp suspension. The mixture was thoroughly stirred with a high speed pigment disintegrator for 10 minutes. Then, under gentle stirring, 2.7 g of polyaluminum chloride and 3.7 g of wet strength resin were added. With this furnish, laboratory handsheets in around 75 g/m² were prepared using the Rapid Köthen method and were used as a reference of the standard recipe.

To evaluate the effect of the polypropylene glycol molecular weight, other furnishes were prepared in the same way, adding 2% polypropylene glycol of different molecular weights (425, 2,000 or 3,500 g/mol).

The results are shown in Table 3.

Furnish D: 75 g of eucalyptus pulp were disintegrated in 2 liters of water. The pulp suspension was then diluted with 2 liters of water. 10 ml of NaOH 3% solution and 52.5 g of titanium dioxide were added to the pulp suspension. The mixture was thoroughly stirred with a high speed pigment disintegrator for 10 minutes. Then, under gentle stirring, 2.7 g of polyaluminum chloride and 3.7 g of wet strength resin were added. With this furnish, laboratory handsheets in around 75 g/m² were prepared using the Rapid Köthen method and were used as a reference of the standard recipe.

To evaluate the effect of the addition of copolymers of propylene glycol and ethylene glycol, other furnishes were prepared in the same way, adding 2% of 2 different polypropylene glycol-block-polyethylene glycol copolymers. Both copolymers had 10% of polyethylene glycol in the molecule but different molecular weights in the polypropylene glycol block: copolymer A, 1,750 g/mol, and copolymer B, 3,250 g/mol.

The results are shown in Table 4.

TABLE 1

| Composition | Standard | Modified | | |
|---|---|---|---|---|
| Polypropylene glycol | 0 | 0.3 | 0.6 | 1.2 |
| (% referred to total solids) | | | | |
| ⁽¹⁾Gurley (s) | 10.3 | 9.6 | 7.8 | 7.7 |
| ⁽²⁾Klemm absorption (mm) | 25-26 | 26-27 | 28-29 | 29-30 |
| Resin penetration (s) | 7.1 | 6 | 5.2 | 5 |
| (MW550 melamine resin solution; 55% | | | | |
| solids) | | | | |
| ⁽³⁾Basis weight (g/m²) | 76.5 | 76.7 | 74.5 | 79 |
| ⁽⁴⁾Ash content (%) | 33 | 32.1 | 31.8 | 32.8 |

TABLE 2

| Composition | Standard | Modified | | |
|---|---|---|---|---|
| Polypropylene glycol | 0 | 0.5 | 1.1 | 2.1 |
| (% referred to total solids) | | | | |
| ⁽¹⁾Gurley (s) | 16.8 | 15.4 | 10.2 | 10.1 |
| ⁽²⁾Klemm absorption (mm) | 20-21 | 20-21 | 22 | 24 |
| Resin penetration (s) | 10.7 | 9.8 | 4.8 | 4.6 |
| (MW550 melamine resin solution; 55% | | | | |
| solids) | | | | |
| ⁽³⁾Basis weight (g/m²) | 82.8 | 83.4 | 82.8 | 82.2 |
| ⁽⁴⁾Ash content (%) | 39.6 | 38.6 | 40.1 | 38.3 |

TABLE 3

| Composition | Standard | Modified | | |
|---|---|---|---|---|
| Polypropylene glycol | — | 425 | 2,000 | 3,500 |
| molecular weight (g/mol) | | | | |
| ⁽¹⁾Gurley (s) | 13 | 9.9 | 8.6 | 8.4 |
| ⁽²⁾Klemm absorption (mm) | 28 | 28-29 | 30 | 29 |
| Resin penetration (s) | 7.0 | 5.0 | 4.5 | 4.4 |
| (MW550melamine resin solution; | | | | |
| 55% solids) | | | | |
| ⁽³⁾Basis weight (g/m²) | 80 | 80.4 | 79.8 | 79 |
| ⁽⁴⁾Ash content (%) | 29.6 | 30 | 29.4 | 30.1 |

TABLE 4

| Composition | Standard | Modified | |
|---|---|---|---|
| Copolymer | — | A | B |
| ⁽¹⁾Gurley (s) | 13.2 | 10 | 11 |
| ⁽²⁾Klemm absorption (mm) | 26 | 28 | 27 |
| Resin penetration (s) | 9.0 | 6.5 | 7.5 |
| (MW550melamine resin solution; 55% | | | |
| solids) | | | |
| ⁽³⁾Basis weight (g/m²) | 80 | 80.4 | 80.6 |
| ⁽⁴⁾Ash content (%) | 28.6 | 28.4 | 29.0 |

⁽¹⁾Based on ISO 5636/5
⁽²⁾Based on DIN ISO 8787
⁽³⁾DIN EN ISO 536
⁽⁴⁾ISO 2144

The results with these four furnish examples show that the addition of polypropylene glycol or copolymers of propylene glycol and ethylene glycol increases paper porosity and Klemm capillary absorption, and decreases melamine resin penetration time.

### EXAMPLE 2

This example shows the effect of polyropylene glycol in decorative paper produced in a Fourdrinier papermaking machine for two different furnish compositions.

Furnish E: eucalyptus pulp was disintegrated in water at pH=9 in the presence of titanium dioxide (50% referred to cellulose). After refining, pH was adjusted in the mixing chest down to 7 with aluminum salts. Then, wet strength resin was added (2% referred to cellulose). With this furnish, decorative paper in 75 g/m² was produced in a conventional papermaking machine.

To evaluate the effect of polypropylene glycol (molecular weight around 2,000 g/mol) in the composition, this polymer was added in continuous at the constant part, adjusting the pumping in order to have from 0.05% to 0.5% of said polymer in the paper produced.

The results are shown in Table 5.

The decorative papers thus produced were impregnated at a pilot impregnation machine. The paper produced according to the modified composition showed faster impregnation properties.

Furnish F: eucalyptus pulp was disintegrated in water at pH=9 in the presence of titanium dioxide (50% referred to cellulose). After refining, pH was adjusted in the mixing chest down to 7 with aluminum salts. Then, wet strength resin was added (2% referred to cellulose). With this furnish, decorative paper in 75 g/m² was produced in a conventional papermaking machine.

To evaluate the effect of polypropylene glycol (molecular weight around 2,000 g/mol) in the composition, 0.15% of said polymer was added to the thick stock, in discontinuous way.

The results are shown in Table 6.

The decorative papers thus produced were impregnated at a pilot impregnation machine. The paper produced according to the modified composition showed faster impregnation properties.

TABLE 5

| Composition | Standard | Modified | | |
|---|---|---|---|---|
| Polypropylene glycol | 0 | 0.15 | 0.21 | 0.47 |
| (% referred to total solids) | | | | |
| ⁽¹⁾Gurley (s) | 14.0 | 8.0 | 7.4 | 5.8 |
| ⁽²⁾Klemm absorption (mm) | 23 | 29 | 30 | 32 |
| Resin penetration (s) | 9.8 | 5.4 | 5.1 | 4.5 |
| (MW550 melamine resin solution; | | | | |
| 55% solids) | | | | |
| ⁽³⁾Basis weight (g/m²) | 78 | 77 | 78 | 77 |
| ⁽⁴⁾Ash content (%) | 27 | 28.5 | 28 | 26.6 |
| Cross direction wet expansion (%) | 1.45 | 1.3 | 1.3 | 1.25 |

TABLE 6

| Composition | Standard | Modified |
|---|---|---|
| Polypropylene glycol | 0 | 0.15 |
| (% referred to total solids) | | |
| ⁽¹⁾Gurley (s) | 16.6 | 13.1 |
| ⁽²⁾Klemm absorption (mm) | 20 | 21 |
| Resin penetration (s) | 6.9 | 5.5 |
| (MW550 melamine resin solution; 55% solids) | | |
| ⁽³⁾Basis weight (g/m²) | 90 | 90 |
| ⁽⁴⁾Ash content (%) | 42.3 | 42 |
| Mutek CD wet expansion (%) | 1.3 | 1.2 |

(1) Based on ISO 5636/5
(2) Based on DIN ISO 8787
(3) DIN EN ISO 536
(4) ISO 2144

The results with both furnishes show that the addition of polypropylene glycol to a standard recipe for producing decorative paper in a Fourdrinier papermaking machine increases paper porosity and capillary absorption, decreases melamine resin penetration time and reduces wet expansion in cross direction. Moreover, the quantities of polymer needed to obtain the same results as in laboratory handsheets are substantially lower.

1. Use of an additive comprising:
a member selected from the group consisting of a polyether of an alkylene glycol and a copolyether of different alkylene glycols, for the production of decorative paper.

2. Use of the additive according to claim 1, comprising:
a member selected from the group consisting of a polypropylene glycol and a copolymer of ethylene glycol and propylene glycol.

3. Use of the additive according to claim 1, comprising:
polypropylene with a molecular weight between 100 g/mol and 10,000 g/mol.

4. Use of the additive according to claim 1, comprising:
poly (ethylene glycol-co-propylene glycol) with a molecular weight between 100 g/mol and 10,000 g/mol, and an ethylene glycol/propylene glycol ratio from 99/1 to 1/99.

5. Use of the additive according to claim 1, wherein:
the additive is added to a cellulosic fibrous furnish before sheet forming on a paper machine wire.

6. Use of the additive according to claim 5, wherein:
the additive is added continuously.

7. Use of the additive according to claim 5, wherein:
the additive is added discontinuously.

8. Use of the additive according to claim 5, wherein:
the additive is incorporated in bulk to the cellulosic fibrous furnish in quantities between 0.05% and 3.0% by weight, referred to the total solids content.

9. Use of the additive according to claim 1, wherein:
the additive is applied on the surface of the decorative paper.

10. Use of the additive according to claim 9, wherein:
the additive is applied by coating.

11. Use of the additive according to claim 9, wherein:
the additive is applied by impregnation.

12. Use of the additive according to claim 1, wherein:
the decorative paper is impregnated (soaked) with the additive.

13. Decorative paper produced with the use of an additive comprising:
a member selected from the group consisting of a polyether of an alkylene glycol and a copolyether of a different alkylene glycols.

14. Use of the additive according to claim 1, comprising:
polypropylene glycol with a molecular weight between 2,000 g/mol and 4,000 g/mol.

15. Use of the additive according to claim 1, comprising:
poly (ethylene glycol-co-propylene glycol) with a molecular weight between 2,000 g/mol and 5,000 g/mol and an ethylene glycol/propylene glycol ratio from 50/50 and 1/99.

16. Use of the additive according to claim 5, wherein:
the additive is incorporated in bulk to the cellulosic fibrous furnish in quantities between 0.25% and 2% by weight, referred to the total solids content.
